(19) United States
(12) Patent Application Publication
(10) Pub. No.: US 2025/0344939 A1
(43) Pub. Date: Nov. 13, 2025

(54) ULTRASONIC ENDOSCOPE

(71) Applicant: FUJIFILM Corporation, Tokyo (JP)

(72) Inventors: Karin HATTORI, Kanagawa (JP); Masaya INOUE, Kanagawa (JP); Yasuhiko MORIMOTO, Kanagawa (JP)

(73) Assignee: FUJIFILM Corporation, Tokyo (JP)

(21) Appl. No.: 19/189,273

(22) Filed: Apr. 25, 2025

(30) Foreign Application Priority Data
May 10, 2024 (JP) 2024-077272

(51) Int. Cl. A61B 1/00

(52) U.S. Cl. CPC A61B 1/00098; A61B 1/00082; A61B 1/00096; A61B 2560/0462; A61B 2562/0204

(57) ABSTRACT

An ultrasonic endoscope includes: a distal end portion disposed on a distal end side of an insertion part extending along a Y direction; an ultrasonic transducer that emits an ultrasonic wave toward a Z(+) direction side in a Z direction; an elevator disposed on a Y(−) direction side with respect to the ultrasonic transducer and rotatable between an elevating position and a falling position; and an observation window disposed on the Y(−) direction side with respect to the elevator. In the Z direction, end portions of the observation window and the elevator on the Z(+) direction side in the Z direction in a case in which the elevator is located at the falling position, and an end portion of the ultrasonic transducer on the Z(+) direction side in the Z direction are disposed in order from the Z(+) direction side.

### CROSS-REFERENCE TO RELATED APPLICATION

The present application claims priority under 35 U.S.C § 119 (a) to Japanese Patent Application No. 2024-077272 filed on May 10, 2024, which is hereby expressly incorporated by reference, in its entirety, into the present application.

### BACKGROUND OF THE INVENTION

### 1. Field of the Invention

The present invention relates to an ultrasonic endoscope, and more particularly to an ultrasonic endoscope comprising an ultrasonic transducer provided at a distal end portion of an insertion part.

### 2. Description of the Related Art

As the ultrasonic endoscope, an ultrasonic endoscope is known which comprises a convex type ultrasonic transducer at a distal end portion of an insertion part, in which a treatment tool outlet port is disposed on a base end side of the ultrasonic transducer at the distal end portion (for example, see WO2020/179909A).

In an examination using the above-described ultrasonic endoscope, for example, while acquiring an ultrasonic image of a treatment target part by an ultrasonic transducer, a biopsy needle (treatment tool) led out into a subject body through a treatment tool insertion channel and a treatment tool outlet port is punctured into the treatment target part to collect cells. In this case, the treatment tool is treated at a desired position by changing a leading-out direction by an elevating and falling operation of an elevator housed in an elevator housing portion.

The distal end portion of the insertion part is provided with an observation window for observing a treatment target part, an illumination window for emitting illumination light toward the treatment target part, and an air and water supply nozzle for jetting cleaning water or air toward the observation window or the like, in addition to the ultrasonic transducer and the elevator.

### SUMMARY OF THE INVENTION

In general, in the distal end portion of the ultrasonic endoscope, the ultrasonic transducer, the elevator, and the observation window are disposed in this order from the distal end side to the base end side, and at least a part of each of the ultrasonic transducer and the elevator is reflected in an endoscopic image taken in from the observation window. As a result, the improvement in the visibility of the biopsy needle and the puncture stability due to the reduction in a puncture distance of the biopsy needle are achieved.

However, in the above-described disposition configuration, the ultrasonic transducer and the elevator are disposed to be close to each other, so that it is necessary to prevent the biopsy needle led out by being guided by the elevator from coming into contact with the ultrasonic transducer. Therefore, it is necessary to increase an inclined angle of the elevator at a falling position of the elevator, but there is a problem in that it is not possible to satisfy a demand to perform the puncture at a small puncture angle (shallow angle) in a case in which such a configuration is adopted.

The present invention has been made in view of such circumstances, and an object of the present invention is to provide an ultrasonic endoscope that enables a puncture at a small puncture angle.

A first aspect of the present invention relates to an ultrasonic endoscope comprising: a distal end portion that is disposed on a distal end side of an insertion part extending along a longitudinal axis direction; an ultrasonic transducer that is provided at the distal end portion and that emits an ultrasonic wave toward one side in a first direction orthogonal to the longitudinal axis direction; an elevator that is disposed on a base end side in the longitudinal axis direction with respect to the ultrasonic transducer and that is provided to be rotatable between an elevating position and a falling position; and an observation window that is disposed on the base end side in the longitudinal axis direction with respect to the elevator and through which an inside of a subject is observed, in which, in the first direction, an end portion of the observation window on the one side in the first direction, an end portion of the elevator on the one side in the first direction in a case in which the elevator is located at the falling position, and an end portion of the ultrasonic transducer on the one side in the first direction are disposed in order from the one side.

A second aspect of the present invention relates to the ultrasonic endoscope according to the first aspect, in which the distal end portion includes a first region in which the ultrasonic transducer is provided and a second region that is disposed on the base end side in the longitudinal axis direction with respect to the first region and in which the elevator and the observation window are provided, a first bottom surface portion of the first region and a second bottom surface portion of the second region are each disposed on the other side in the first direction, which is opposite to the one side, and the first bottom surface portion is disposed on the other side in the first direction with respect to the second bottom surface portion.

A third aspect of the present invention relates to the ultrasonic endoscope according to the second aspect, in which, in a case of being viewed in a second direction orthogonal to each of the longitudinal axis direction and the first direction, an angle formed between a first central axis of the first region and a second central axis of the second region is 1 degree or less.

A fourth aspect of the present invention relates to the ultrasonic endoscope according to the third aspect, in which, in a case of being viewed in the second direction, an angle formed between a plane direction of the first bottom surface portion and the longitudinal axis direction is 1 degree or less.

A fifth aspect of the present invention relates to the ultrasonic endoscope according to any one of the second to fourth aspects, in which the distal end portion includes a balloon groove that is provided between the first region and the second region and that is used for attaching a balloon that covers the ultrasonic transducer.

A sixth aspect of the present invention relates to the ultrasonic endoscope according to the fifth aspect, in which a groove region of the balloon groove on the other side in the first direction is formed of a pair of groove side surface portions facing each other in the longitudinal axis direction, a first groove side surface portion that is one of the pair of groove side surface portions is provided on a base end side of the first region in the longitudinal axis direction, and a second groove side surface portion that is the other of the pair of groove side surface portions is provided on a distal end side of the second region in the longitudinal axis direction, and the first groove side surface portion is formed to extend toward the other side in the first direction with respect to the second groove side surface portion.

A seventh aspect of the present invention relates to the ultrasonic endoscope according to any one of the first to sixth aspects, in which the ultrasonic transducer is a convex type in which a plurality of ultrasonic oscillators are arranged in an arc shape along the longitudinal axis direction, and the elevator is disposed outside an ultrasonic wave scanning range of the ultrasonic transducer.

An eighth aspect of the present invention relates to the ultrasonic endoscope according to any one of the first to seventh aspects, in which the ultrasonic wave scanning range of the ultrasonic transducer is 180 degrees or less.

According to the aspects of the present invention, it is possible to perform the puncture at a small puncture angle.

### BRIEF DESCRIPTION OF THE DRAWINGS

FIG. 1 is an overall configuration diagram of an ultrasonic endoscope according to the present embodiment.

FIG. 2 is a perspective view showing an appearance of a distal end member.

FIG. 3 is a side view of the distal end member showing a puncture direction of a biopsy needle.

FIG. 4 is a side view of the distal end member showing heights of a first region and a second region.

FIGS. 5A to 5C are explanatory diagrams showing another configuration of the distal end member.

FIG. 6 is an enlarged view of a balloon groove.

FIG. 7 is a side view of the distal end member showing an ultrasonic wave scanning range.

### DESCRIPTION OF THE PREFERRED EMBODIMENTS

Hereinafter, an embodiment of the present invention will be described with reference to the accompanying drawings.

### Overall Configuration of Ultrasonic Endoscope

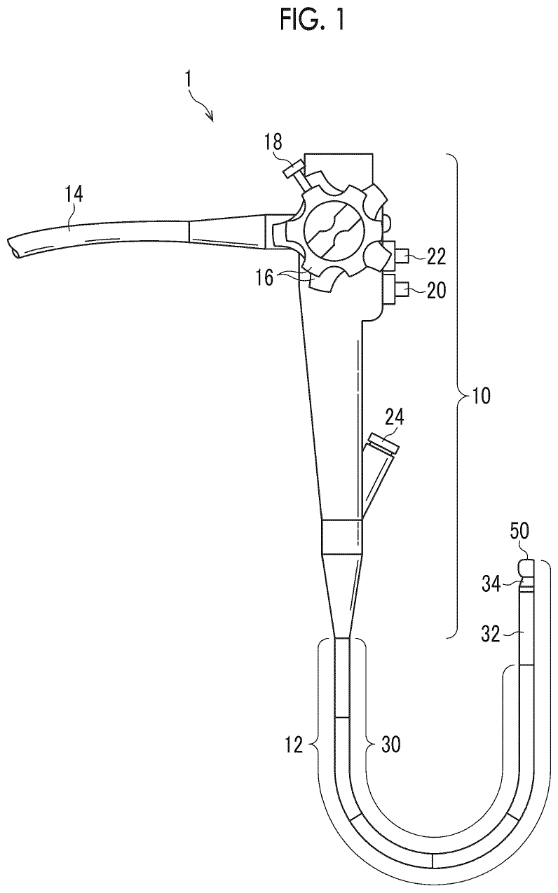

FIG. 1 is an overall diagram of an ultrasonic endoscope (hereinafter, abbreviated as an “endoscope”) 1 according to the embodiment of the present invention.

As shown in FIG. 1, the endoscope 1 according to the present embodiment comprises an operating part 10 that is gripped by an operator to perform various operations, an insertion part 12 that is inserted into a body cavity of a patient, and a universal cord 14. The endoscope 1 is connected to a system constituent device including a processor device and a light source device (not shown) through the universal cord 14.

The operating part 10 is provided with various operation members operated by the operator and, for example, is provided with a pair of angle knobs 16, an elevating operation lever 18, an air and water supply button 20, a suction button 22, and the like.

In addition, a treatment tool inlet port 24 is provided on a distal end side of the operating part 10. The treatment tool introduced from the treatment tool inlet port 24 is inserted into a treatment tool insertion channel inserted into the insertion part 12.

The insertion part 12 extends from a distal end of the operating part 10 along a longitudinal axis direction, and the entire insertion part 12 is formed to have a small diameter and a long shape. The insertion part 12 includes a soft portion 30, a bendable portion 32, and a distal end portion 34 in order from a base end side to a distal end side. It should be noted that the insertion part 12 is an example of an insertion part according to the embodiment of the present invention, and the distal end portion 34 is an example of a distal end portion according to the embodiment of the present invention.

The soft portion 30 occupies most of the insertion part 12 from the base end side and has enough flexibility to be bent in any direction. In a case in which the insertion part 12 is inserted into the body cavity, the soft portion 30 is bent along an insertion route in the body cavity.

The bendable portion 32 is bent in an up-down direction and a left-right direction by a rotational movement operation of the pair of angle knobs 16 of the operating part 10. The distal end portion 34 can be directed in a desired direction by performing the bending operation of the bendable portion 32.

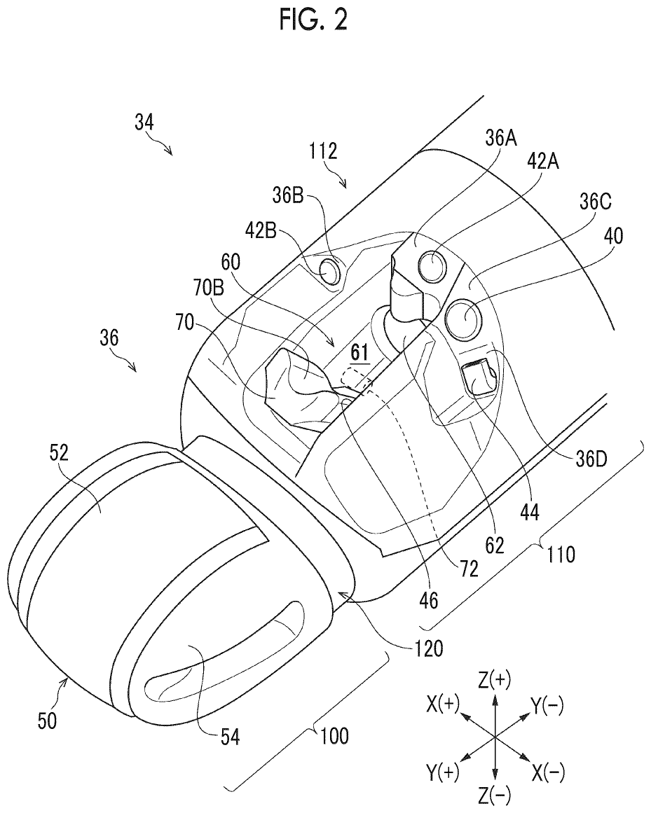

The distal end portion 34 comprises a distal end member 36 (see FIG. 2) described later. The distal end member 36 is disposed on the distal end side of the insertion part 12. The distal end member 36 includes an ultrasonic observation portion 100 on the distal end side thereof and an endoscope observation portion 110 on the base end side of the ultrasonic observation portion 100.

The universal cord 14 shown in FIG. 1 encompasses built-in components such as an ultrasonic cable, an electric cable, a light guide, and a fluid tube. A connector is provided at an end portion of the universal cord 14 (not shown), and by connecting the connector to the system constituent device, a control signal, power, illumination light, liquid, gas, and the like necessary for the operation of the endoscope 1 are supplied from the system constituent device to the endoscope 1. It should be noted that, in addition to the built-in components, a built-in component such as a treatment tool insertion channel, a bending operation wire, and an elevator operation wire is inserted into the insertion part 12.

Data of an ultrasonic image and data of an endoscopic image acquired by the endoscope 1 (ultrasonic observation portion 100 and endoscope observation portion 110) are each transmitted from the endoscope 1 to the system constituent device. Each data transmitted to the system constituent device is processed by the system constituent device and then displayed on a monitor (not shown) as the ultrasonic image and the endoscopic image, respectively.

Next, a configuration of the distal end portion 34 (distal end member 36) will be described with reference to FIG. 2. FIG. 2 is a perspective view showing an appearance of the distal end member 36, and shows a state in which an elevator 70 to be described later is located at a falling position.

Hereinafter, in a case of describing the configuration of each portion of the distal end member 36, a three-dimensional orthogonal coordinate system of X, Y, and Z will be used for convenience of description. In the drawings, a Z direction indicates an up-down direction, a Z(+) direction side indicates an up direction, and a Z(−) direction side indicates a down direction. In addition, in the drawings, an X direction indicates a direction perpendicular to the Z direction, an X(+) direction side indicates a left direction, and an X(−) direction side indicates a right direction. In addition, in the drawings, a Y direction indicates a direction perpendicular to both the Z direction and the X direction, a Y(+) direction side indicates a distal end side direction, and a Y(−) direction side indicates a base end side direction. It should be noted that each of the above-described directions indicates a direction in a case in which the distal end member 36 is viewed from the distal end side and an ultrasonic wave transmission and reception surface 52 of an ultrasonic transducer 50 described later is directed upward.

In addition, the Y direction corresponds to a longitudinal axis direction of the insertion part according to the embodiment of the present invention (hereinafter, may be simply referred to as a “longitudinal axis direction”). In addition, the Z direction corresponds to a first direction according to the embodiment of the present invention, the Z(+) direction side corresponds to one side in the first direction according to the embodiment of the present invention, and the Z(−) direction side corresponds to the other side in the first direction according to the embodiment of the present invention.

As shown in FIG. 2, the distal end member 36 includes the ultrasonic observation portion 100 and the endoscope observation portion 110 disposed on the Y(−) direction side (base end side) of the ultrasonic observation portion 100.

The ultrasonic observation portion 100 and the endoscope observation portion 110 are consecutively installed through a balloon groove 120 formed between the ultrasonic observation portion 100 and the endoscope observation portion 110. The balloon groove 120 is formed over the entirety of a circumferential direction around the Y direction (longitudinal axis direction). A balloon that covers the ultrasonic transducer 50 is attached to the balloon groove 120. It should be noted that the balloon groove 120 is an example of a balloon groove according to the embodiment of the present invention.

The ultrasonic observation portion 100 includes the ultrasonic transducer 50, and the ultrasonic transducer 50 is held by a housing 54.

The ultrasonic transducer 50 is a convex type in which a plurality of ultrasonic oscillators are arranged in an arc shape along the Y direction (longitudinal axis direction). The ultrasonic transducer 50 is configured such that an upper side surface (surface on the Z(+) direction side) thereof is formed as an ultrasonic wave transmission and reception surface 52, and emits an ultrasonic wave from the ultrasonic wave transmission and reception surface 52 toward the Z(+) direction side (one side in the first direction). Specifically, in a case of being viewed in the X direction, the ultrasonic waves emitted from the ultrasonic wave transmission and reception surface 52 (plurality of ultrasonic oscillators) are fan-shaped scanned (convex-scanned) obliquely rearward (Y(−) direction side and Z(+) direction side) to obliquely forward (Y(+) direction side and Z(+) direction side). The emission direction of the ultrasonic waves scanned in this way includes at least a component on the Z(+) direction side. It should be noted that it is sufficient that the emission direction of most of the ultrasonic waves to be scanned include the component on the Z(+) direction side, and the emission direction of some ultrasonic waves need not include the component on the Z(+) direction side (for example, in a case of the Y(+) direction side or in a case in which the direction includes a component on the Z(−) direction side). Data for generating the ultrasonic image is acquired by the ultrasonic transducer 50 configured in this manner. It should be noted that the ultrasonic transducer 50 is provided at the distal end portion 34 and is an example of an ultrasonic transducer according to the embodiment of the present invention.

The endoscope observation portion 110 includes a body member 112 formed in a substantially cylindrical shape. The body member 112 is made of an insulating material having insulating properties, for example, a resin material such as plastic materials such as a methacrylic resin, a polyphenyl sulfone resin, a polyether imide resin, a polyether ether ketone resin, and a polycarbonate.

The body member 112 includes an observation window 40 for observing an inside of a subject, illumination windows 42A and 42B for illuminating the inside of the subject, and an air and water supply nozzle 44 for cleaning the observation window 40 and the like. The observation window 40 is disposed on the Y(−) direction side (base end side) with respect to the elevator 70. It should be noted that the observation window 40 is an example of an observation window according to the embodiment of the present invention.

The body member 112 includes an elevator housing portion 60 that houses the elevator 70 therein. The elevator housing portion 60 includes an opening 46 having a rectangular shape in plan view in a case of being viewed from the Z(+) direction side. The opening 46 is formed to be open toward the Z(+) direction side in the body member 112. An opening direction of the opening 46 in the present embodiment is a direction (that is, a direction perpendicular to an X-Y plane) including only a component on the Z(+) direction side in a case of being viewed in the X direction. It should be noted that the opening direction of the opening 46 need only be a direction including at least the component on the Z(+) direction side, and may be, for example, a direction (that is, a direction including both components on the Z(+) direction side and the Y(+) direction side), which is obliquely forward in a case of being viewed in the X direction.

A treatment tool such as a biopsy needle is led out of the distal end member 36 from the opening 46. This treatment tool is led out toward an ultrasonic wave scanning range of the ultrasonic transducer 50. In addition, the elevator housing portion 60 is provided on a lower side (Z(−) direction side) of the opening 46.

A treatment tool outlet port 62 that communicates with an elevator housing space 61 of the elevator housing portion 60 is provided on the Y(−) direction side (base end side) of the elevator housing portion 60. The treatment tool outlet port 62 communicates with the treatment tool inlet port 24 of the operating part 10 via the treatment tool insertion channel inserted and disposed into the insertion part 12 (see FIG. 1). As a result, the treatment tool introduced from the treatment tool inlet port 24 is guided to the elevator housing space 61 from the treatment tool outlet port 62 via the treatment tool insertion channel.

The elevator 70 is housed in the elevator housing space 61 of the elevator housing portion

60. The elevator 70 is disposed on the Y(−) direction side with respect to the ultrasonic transducer 50. The elevator 70 is provided in a rotationally movable manner about a rotational movement shaft 72 disposed along the X direction, and is rotationally moved between an elevating position and a falling position. The elevating position refers to a position of the elevator 70 (that is, a position at which the elevator 70 is in a state of being elevated toward the Z(+) direction side) in a case in which the distal end side (opposite side to the rotational movement shaft 72) of the elevator 70 is moved to an end position on the Y(−) direction side in a rotationally movable range of the elevator 70. The falling position refers to a position of the elevator 70 (that is, a position at which the elevator 70 is in a state of being fallen toward the Y(+) direction side) in a case in which the distal end side of the elevator 70 is moved to an end position on the Y(+) direction side in the rotationally movable range of the elevator 70.

The elevator 70 is made of a metal material such as stainless steel, and includes a treatment tool guide surface 70B on the upper surface side. The treatment tool guided to the elevator housing portion 60 is guided along the treatment tool guide surface 70B, and is led out of the opening 46 of the elevator housing portion 60.

In a case in which the elevating operation lever 18 (see FIG. 1) is operated, the elevator 70 configured as described above operates (elevating and falling operation) between the elevating position and the falling position about the rotational movement shaft 72. For example, a leading-out direction (leading-out angle) of the treatment tool guided by the treatment tool guide surface 70B of the elevator 70 and led out from the opening 46 can be changed by operating the elevator 70 by the operation of the elevating operation lever 18 and adjusting an elevating angle of the elevator 70 from the falling position. It should be noted that the elevator 70 is an example of an elevator according to the embodiment of the present invention.

As shown in FIG. 2, the illumination windows 42A and 42B are disposed respectively on illumination window disposition surfaces 36A and 36B provided on the body member 112. Inside the illumination windows 42A and 42B, light emission portions constituting an illumination unit are housed. These light emission portions are connected to a light source device via a light guide. The illumination light transmitted from the light source device through the light guide is emitted from the light emission portions and to irradiate a treatment target part through the illumination windows 42A and 42B.

The observation window 40 is disposed on an observation window disposition surface 36C provided in the body member 112. An imaging system unit including an imaging optical system, a solid-state imaging element, and a circuit board, which constitute an imaging unit (camera unit), is housed in the observation window 40. Accordingly, light (reflected light) from the treatment target part irradiated by the illumination windows 42A and 42B is taken in from the observation window 40, and the light is imaged as an observation image on the solid-state imaging element through the imaging optical system. Data for generating the endoscopic image is acquired by the imaging unit. It should be noted that the observation window 40 is an example of an observation window according to the embodiment of the present invention.

The air and water supply nozzle 44 is disposed on a nozzle disposition surface 36D provided on the body member 112. In a case in which the air and water supply button 20 (see FIG. 1) is operated, a cleaning liquid such as water or air (fluid) is jetted from the air and water supply nozzle 44 toward the observation window 40 or the like, to clean the observation window 40 or the like.

It should be noted that, in addition to the imaging unit, an ultrasonic cable connected to the ultrasonic transducer 50, an elevator unit, and the like are disposed inside the body member 112. The elevator unit is a unit in which the elevator 70, an elevator support portion that supports the elevator 70 in a rotationally movable manner, and a drive mechanism for rotationally moving the elevator 70 are formed as an integrated component, and the elevator unit constitutes the elevator housing portion 60.

In the ultrasonic endoscope, there is a demand for puncturing the treatment target part with the biopsy needle at a shallow puncture angle while avoiding contact with the ultrasonic transducer. Therefore, the endoscope 1 according to the present embodiment has the following configuration in order to satisfy the above-described demand.

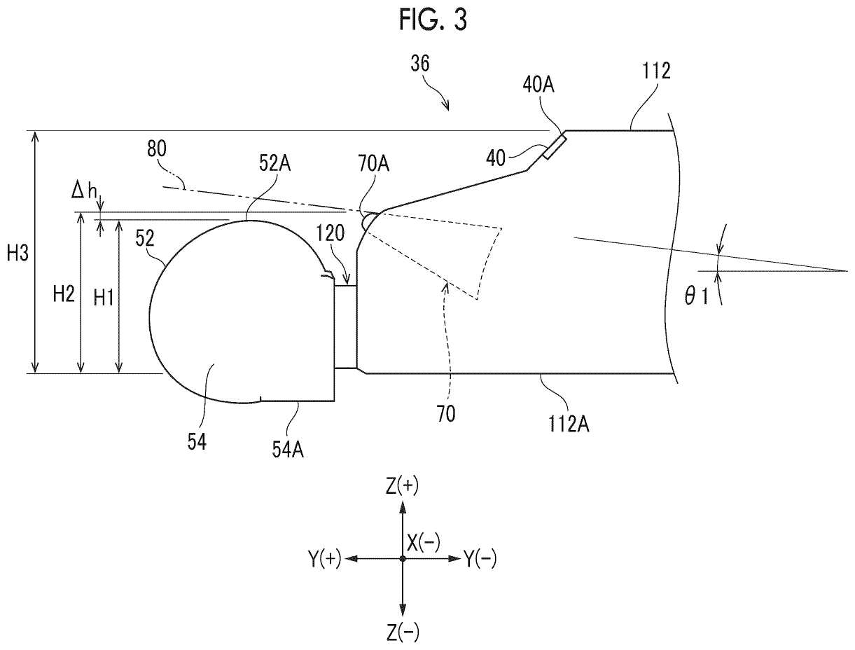

FIG. 3 is a side view in a case in which the distal end member 36 is viewed from the X(−) direction side. In FIG. 3, the elevator 70 is located at the falling position, and a biopsy needle 80 guided and led out by the elevator 70 is indicated by a two-dot chain line.

As shown in FIG. 3, in the endoscope 1 according to the present embodiment, the ultrasonic transducer 50, the elevator 70, and the observation window 40 are disposed in order from the Y(+) direction side (distal end side) to the Y(−) direction side (base end side) in the distal end member 36.

In the disposition configuration as described above, in the present embodiment, in the Z direction, an end portion of the observation window 40 on the Z(+) direction side in the Z direction, an end portion of the elevator 70 on the Z(+) direction side in the Z direction in a case in which the elevator 70 is located at the falling position, and an end portion of the ultrasonic transducer 50 on the Z(+) direction side in the Z direction are disposed in order from the Z(+) direction side.

In addition, regarding the above-described disposition configuration, for example, in a case in which a position of a bottom surface portion 112A of the body member 112 in the Z direction is set as a reference position of a height, and a direction from the reference position toward the Z(+) direction side in the Z direction is set as a height direction, it can be said that the ultrasonic transducer 50, the elevator 70, and the observation window 40 have the following height relationship.

That is, in a case in which a height of a top portion 52A of the ultrasonic wave transmission and reception surface 52, which is the highest position of the ultrasonic transducer 50 (distance from the bottom surface portion 112A to the top portion 52A), is denoted by a first height H1, a height of the distal end portion 70A of the elevator 70, which is the highest position of the elevator 70 in a case in which the elevator 70 is located at the falling position (distance from the bottom surface portion 112A to the distal end portion 70A), is denoted by a second height H2, and a height of an upper edge portion 40A of the observation window 40, which is the highest position of the observation window 40 (distance from the bottom surface portion 112A to the upper edge portion 40A), is denoted by a third height H3, the second height H2 has a relationship (H1<H2<H3) in which the second height H2 is higher than the first height H1 and lower than the third height H3.

It should be noted that the respective heights H1, H2, and H3 are shown as heights in a case in which the bottom surface portion 112A of the body member 112 is set as the reference position (reference height) as an example, but the present invention is not limited to this, and any position in the Z direction may be used as a reference. For example, a bottom surface portion 54A of the housing 54 may be set as the reference position.

As described above, in the endoscope 1 according to the present embodiment, the positions of the end portions of the ultrasonic transducer 50, the elevator 70, and the observation window 40 on the Z(+) direction side in the Z direction satisfy the above-described relationship. With this configuration, a part of each of the biopsy needle 80 and the ultrasonic transducer 50 can be reflected in the endoscopic image taken in from the observation window 40. As a result, the improvement in the visibility of the biopsy needle 80 and the puncture stability due to the reduction in a puncture distance of the biopsy needle 80 can be achieved.

In addition, in the endoscope 1 according to the present embodiment, the end portion of the ultrasonic transducer 50 on the Z(+) direction side in the Z direction is located on the Z(−) direction side with respect to the end portion of the elevator 70 on the Z(+) direction side in the Z direction in a case in which the elevator 70 is located at the falling position (that is, the first height H1 is lower than the second height H2 (H1<H2)). Due to this relationship, for example, the inclined angle θ1 (inclined angle of the elevator 70 with respect to the Y direction (longitudinal axis direction)) of the elevator 70 at the falling position can be made smaller by an angle corresponding to the distance between the end portions in the Z direction (that is, the difference (Δh(H2−H1)) between the first height H1 and the second height H2) as compared with a case in which the end portion of the ultrasonic transducer 50 on the Z(+) direction side in the Z direction is located on the Z(+) direction side with respect to the end portion of the elevator 70 on the Z(+) direction side in the Z direction in a case in which the elevator 70 is located at the falling position (that is, in a case in which the relationship between the first height H1 and the second height H2 is H1≥H2). As a result, the puncture angle of the biopsy needle 80 guided by the treatment tool guide surface 70B of the elevator 70 can be reduced (made shallower).

Therefore, with the endoscope 1 according to the present embodiment, since the respective positions of the ultrasonic transducer 50, the elevator 70, and the observation window 40 are configured to satisfy the above-described relationship, it is possible to perform the puncture at a small puncture angle.

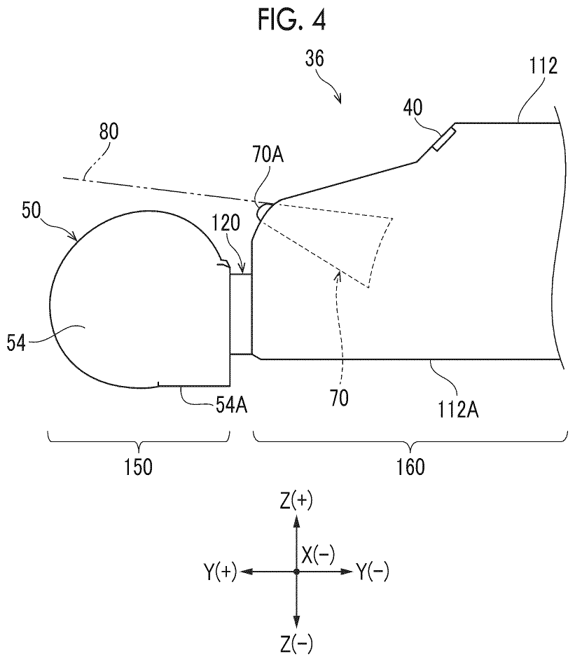

Then, an example of implementing the above-described configuration will be described with reference to FIG. 4. FIG. 4 is a side view in a case in which the distal end member 36 is viewed from the X(−) direction side. It should be noted that FIG. 4 is a diagram in which information indicating a height relationship between the respective portions is deleted from the diagram shown in FIG. 3 for convenience of description.

As shown in FIG. 4, the distal end member 36 includes a first region 150 in which the ultrasonic transducer 50 is provided and a second region 160 that is disposed on the Y(−) direction side (base end side) with respect to the first region 150 and in which the elevator 70 and the observation window 40 are provided.

The first region 150 includes the ultrasonic observation portion 100 (see FIG. 2), and the second region 160 includes the endoscope observation portion 110 (see FIG. 2). It should be noted that the first region 150 and the second region 160 are examples of a first region and a second region according to the embodiment of the present invention, respectively.

In addition, in the distal end member 36, the bottom surface portion 54A of the housing 54 and the bottom surface portion 112A of the body member 112 are disposed on the Z(−) direction side in the Z direction, and the bottom surface portion 54A is disposed on the Z(−) direction side in the Z direction with respect to the bottom surface portion 112A.

Specifically, only the housing 54 disposed on the Y(+) direction side with respect to the balloon groove 120 is disposed to be shifted to the Z(−) direction side with respect to the body member 112, and the bottom surface portion 54A of the housing 54 is disposed to be shifted to the Z(−) direction side by at least Δh (see FIG. 3) with respect to the bottom surface portion 112A of the body member 112.

With the above-described configuration, it is possible to easily achieve a configuration in which the positions of the ultrasonic transducer 50, the elevator 70, and the observation window 40 satisfy the above-described relationship without reducing the size of the ultrasonic transducer 50. As a result, the inclined angle θ1 (see FIG. 3) of the elevator 70 at the falling position can be reduced, and the puncture angle of the biopsy needle 80 can be reduced (made shallow). It should be noted that the bottom surface portion 54A of the housing 54 is an example of a first bottom surface portion of the first region according to the embodiment of the present invention, and the bottom surface portion 112A of the body member 112 is an example of a second bottom surface portion of the second region according to the embodiment of the present invention.

In addition, with the above-described configuration, it is possible to reduce the puncture angle of the biopsy needle 80 without increasing (enlarging) the diameter of the distal end portion 34. Hereinafter, the description will be made with a comparative example.

### Comparative Example

Here, as an example, a distal end portion of the comparative example is a portion in which the height positions of the bottom surface portions 54A and 112A of the housing 54 and the body member 112 are the same in the Z direction.

In the distal end portion of the comparative example, in order to reduce the puncture angle of the biopsy needle 80 without reducing the size of the ultrasonic transducer 50, it is necessary to shift the height position of the rotational movement shaft 72 of the elevator 70 to the Z(+) direction side. In this case, since the inclined angle θ1 (see FIG. 3) of the elevator 70 at the falling position can be reduced while avoiding the contact with the ultrasonic transducer 50, the puncture angle of the biopsy needle 80 can be reduced. However, on the other hand, in the distal end portion of the comparative example, the distal end member 36 is increased in diameter by the amount of shifting of the rotational movement shaft 72 to the Z(+) direction side in order to reduce the puncture angle of the biopsy needle 80, so that the distal end portion is increased in diameter.

### Present Example

The distal end portion 34 of the present example is configured such that the bottom surface portion 54A of the housing 54 is disposed on the Z(−) direction side with respect to the bottom surface portion 112A of the body member 112, as compared to the distal end portion of the comparative example, so that the distal end member 36 is not increased in diameter. As a result, the puncture angle of the biopsy needle 80 can be reduced without increasing the diameter of the distal end portion 34.

Here, from the viewpoint of reducing the burden on the patient and reducing complications, it is desired to reduce the diameter of the distal end portion of the ultrasonic endoscope, but there is a problem in that it is difficult to reduce the diameter of the distal end portion of the ultrasonic endoscope because the ultrasonic endoscope has many functions. In particular, in a case of the ultrasonic endoscope comprising the ultrasonic transducer, it is difficult to reduce the diameter of the distal end portion because large built-in components such as the imaging unit, the ultrasonic cable, and the elevator unit described above has be disposed in the internal space of the distal end portion.

Even in an ultrasonic endoscope in which it is difficult to reduce the diameter of the distal end portion, since the endoscope 1 according to the present embodiment adopts the configuration of the distal end portion 34 in which the bottom surface portion 54A of the housing 54 is disposed on the Z(−) direction side with respect to the bottom surface portion 112A of the body member 112, it is possible to achieve the puncture at a small puncture angle without increasing the diameter of the distal end portion 34.

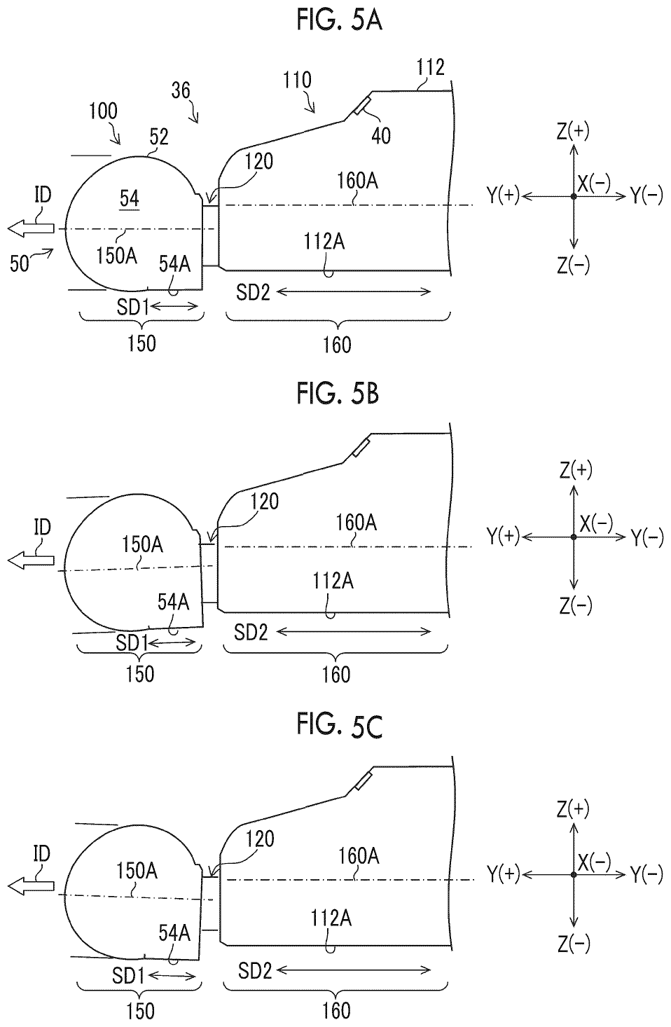

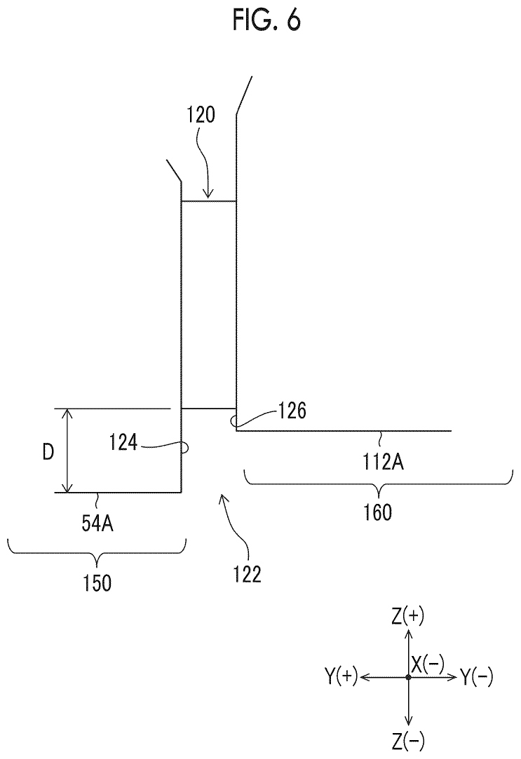

Next, some preferred embodiments of the distal end member 36 of the present example will be described with reference to FIGS. 5A to 6.

### Disposition Relationship between First Region 150 and Second Region 160

FIGS. 5A to 5C is side views in a case in which the distal end member 36 is viewed from the X(−) direction side. As shown in FIGS. 5A to 5C, the first region 150 including the ultrasonic observation portion 100 and the second region 160 including the endoscope observation portion 110 are disposed with the balloon groove 120 interposed therebetween. In addition, as described above, the bottom surface portion 54A of the first region 150 is located on the Z(−) direction side (the other side) with respect to the bottom surface portion 112A of the second region 160. A preferable disposition relationship between the first region 150 and the second region 160 will be described based on respective central axes 150A and 160A.

Here, the central axis 150A is a straight line that passes through the center between the bottom surface portion 54A and a highest point (height direction) of the ultrasonic wave transmission and reception surface 52, and that is parallel to a plane direction SD1 of the bottom surface portion 54A. In addition, the central axis 160A is a straight line that passes through the center of the bottom surface portion 112A and a highest point (height direction) of the body member 112, and that is parallel to a plane direction SD2 of the bottom surface portion 112A. The central axis 150A and the central axis 160A are examples of a first central axis of the first region and a second central axis of the second region according to the embodiment of the present invention, respectively.

In the distal end portion 34, it is preferable that the angle formed between the central axis 150A of the first region 150 and the central axis 160A of the second region 160 is 1 degree or less. By setting the angle to 1 degree or less, in a case in which the distal end portion 34 is inserted into the body cavity, the first region 150 including the ultrasonic observation portion 100 can be prevented from being pressed against the body cavity.

FIGS. 5A, 5B, and 5C show examples of the angle formed between the central axis 150A and the central axis 160A, and the respective angles are different from each other. In FIGS. 5A to 5C, the angle is defined as an angle of the central axis 150A with respect to the central axis 160A, the central axis 160A being parallel to the Y direction.

In FIG. 5A, the central axis 150A and the central axis 160A are each parallel to the Y direction. In this configuration, the angle formed between the central axis 150A and the central axis 160A is 0 degrees.

In FIG. 5B, the central axis 150A is inclined with respect to the Y direction, and the Y(+) direction side is lower than the Y(−) direction side in the Z direction. In this configuration, the angle of the central axis 150A relative to the central axis 160A is 1 degree.

In FIG. 5C, the central axis 150A is inclined with respect to the Y direction, and the Y(+) direction side is higher than the Y(−) direction side in the Z direction. In this configuration, the angle of the central axis 150A relative to the central axis 160A is 1 degree.

In all of FIGS. 5A, 5B, and 5C, the angle between the central axis 150A and the central axis 160A is 1 degree or less.

Then, a preferable relationship between the insertion direction ID and the plane direction SD1 of the bottom surface portion 54A will be described. The insertion direction ID is a direction in which the distal end portion 34 is inserted into the body cavity. The plane direction SD1 of the bottom surface portion 54A is a direction extending along the Y direction as indicated by an arrow. It is preferable that the angle formed between the insertion direction ID and the plane direction SD1 of the bottom surface portion 54A is 1 degree or less. By setting the angle to 1 degree or less, in a case in which the distal end portion 34 is inserted into the body cavity, it is possible to avoid the ultrasonic wave transmission and reception surface 52 or the bottom surface portion 54A from being pressed against the body cavity wall.

It should be noted that, in FIGS. 5A, 5B, and 5C, the angle between the insertion direction ID and the plane direction SD1 of the bottom surface portion 54A is 1 degree or less. FIGS. 5A to 5C show a case in which the central axis 150A and the plane direction SD1 of the bottom surface portion 54A are parallel to each other, but the central axis 150A and the plane direction SD1 of the bottom surface portion 54A need not be parallel to each other.

### Balloon Groove 120

FIG. 6 is an enlarged view of the balloon groove 120 shown in FIG. 3 in a case of being viewed from the X(−) direction side. As shown in FIG. 6, a groove region 122 on the Z(−) direction side of the balloon groove 120 is formed of a pair of groove side surface portions 124 and 126 facing each other the Y direction. Then, among the pair of groove side surface portions 124 and 126, the groove side surface portion 124 is provided on the Y(−) direction side (base end side) of the first region 150, the groove side surface portion 126 is provided on the Y(+) direction side (distal end side) of the second region 160, and the groove side surface portion 124 is formed to extend to the Z(−) direction side with respect to the groove side surface portion 126. It should be noted that the groove side surface portion 124 and the groove side surface portion 126 are examples of a first groove side surface portion and a second groove side surface portion according to the embodiment of the present invention, respectively.

In the attachment of the balloon to the distal end portion 34, in the convex type endoscope 1, since the ultrasonic wave transmission and reception surface 52 is formed of a surface curved in the Z(+) direction side (upper side), a depth of the upper side of the balloon groove 120 does not affect the attachment of the balloon. On the other hand, since the bottom surface portion 54A on the Z(−) direction side (lower side) of the housing 54 is formed of a substantially flat surface, a depth of the balloon groove 120 on the lower side is shallow, and it is difficult to attach the balloon.

However, in the present example, by disposing the bottom surface portion 54A on the Z(−) direction side with respect to the bottom surface portion 112A, the groove side surface portion 124 can be extended on the Z(−) direction side with respect to the groove side surface portion 126. As a result, a depth D of the balloon groove 120 on the Z(−) direction side can be increased, and the attachability of the balloon is improved.

### Disposition Position of Elevator 70

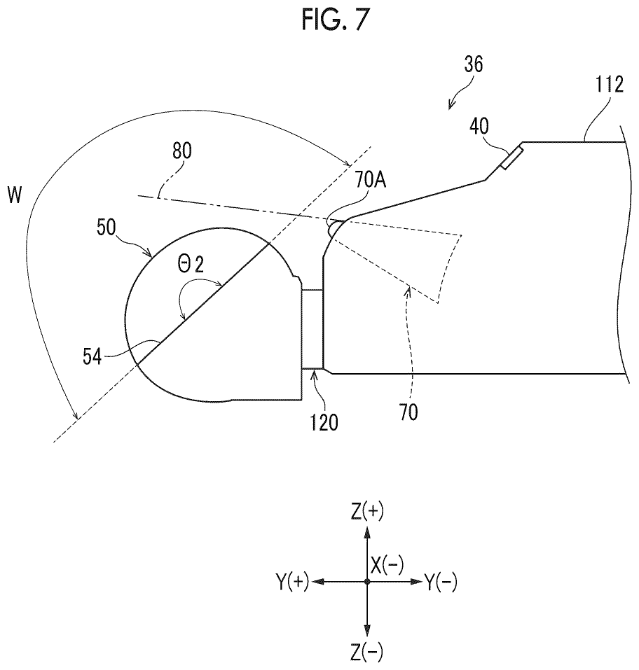

FIG. 7 is a side view of the distal end portion 34 showing an ultrasonic wave scanning range W of the ultrasonic transducer 50.

As shown in FIG. 7, the elevator 70 is disposed outside the ultrasonic wave scanning range W of the ultrasonic transducer 50. It should be noted that, in FIG. 7, the elevator 70 at the falling position is shown, but the elevator 70 is disposed outside the ultrasonic wave scanning range W even at the elevating position.

As a result, it is possible to prevent the elevator 70 from entering the ultrasonic wave scanning range W of the ultrasonic transducer 50. In addition, the ultrasonic wave scanning range W of the ultrasonic transducer 50 shown in FIG. 7 is about 180 degrees, but the ultrasonic wave scanning range W need only be 180 degrees or less, and is, for example, preferably 90 degrees or more. It should be noted that, here, a spread angle θ2 of the ultrasonic waves emitted from the ultrasonic transducer 50 is defined as the ultrasonic wave scanning range W.

### Others

The endoscope 1 according to the present embodiment is for reducing the puncture angle of the biopsy needle 80, in other words, does not require a large puncture angle. Accordingly, the rotational movement angle of the lever built in the body member 112 for rotationally moving the elevator 70 can also be reduced. In a case in which the rotational movement angle of the lever is large, the diameter of the distal end portion 34 is increased accordingly, but, in the present example, since the rotational movement angle of the lever can be reduced, the diameter of the distal end portion 34 can be reduced.

Although the ultrasonic endoscope according to the present embodiment has been described above, the present invention may be improved or modified in some ways without departing from the gist of the present invention.

### EXPLANATION OF REFERENCES

1: ultrasonic endoscope
10: operating part
12: insertion part
14: universal cord
16: angle knob
18: elevating operation lever
20: air and water supply button
22: suction button
24: treatment tool inlet port
30: soft portion
32: bendable portion
34: distal end portion
36: distal end member
36A: illumination window disposition surface
36B: illumination window disposition surface
36C: observation window disposition surface
36D: nozzle disposition surface
40: observation window
40A: upper edge portion
42A: illumination window
42B: illumination window
44: air and water supply nozzle
46: opening
50: ultrasonic transducer
52: ultrasonic wave transmission and reception surface
52A: top portion
54: housing
54A: bottom surface portion
60: elevator housing portion
61: elevator housing space
62: treatment tool outlet port
70: elevator
70A: distal end portion
70B: treatment tool guide surface
72: rotational movement shaft
80: biopsy needle
100: ultrasonic observation portion
110: endoscope observation portion
112: body member
112A: bottom surface portion
120: balloon groove
122: groove region
124: groove side surface portion
126: groove side surface portion
150: first region
160: second region
W: ultrasonic wave scanning range

What is claimed is:

1. An ultrasonic endoscope comprising:
a distal end portion that is disposed on a distal end side of an insertion part extending along a longitudinal axis direction;
an ultrasonic transducer that is provided at the distal end portion and that emits an ultrasonic wave toward one side in a first direction orthogonal to the longitudinal axis direction;
an elevator that is disposed on a base end side in the longitudinal axis direction with respect to the ultrasonic transducer and that is provided to be rotatable between an elevating position and a falling position; and
an observation window that is disposed on the base end side in the longitudinal axis direction with respect to the elevator and through which an inside of a subject is observed,
wherein, in the first direction, an end portion of the observation window on the one side in the first direction, an end portion of the elevator on the one side in the first direction in a case in which the elevator is located at the falling position, and an end portion of the ultrasonic transducer on the one side in the first direction are disposed in order from the one side.

2. The ultrasonic endoscope according to claim 1,
wherein the distal end portion includes a first region in which the ultrasonic transducer is provided and a second region that is disposed on the base end side in the longitudinal axis direction with respect to the first region and in which the elevator and the observation window are provided,
a first bottom surface portion of the first region and a second bottom surface portion of the second region are each disposed on the other side in the first direction, which is opposite to the one side, and
the first bottom surface portion is disposed on the other side in the first direction with respect to the second bottom surface portion.

3. The ultrasonic endoscope according to claim 2,
wherein, in a case of being viewed in a second direction orthogonal to each of the longitudinal axis direction and the first direction, an angle formed between a first central axis of the first region and a second central axis of the second region is 1 degree or less.

4. The ultrasonic endoscope according to claim 3,
wherein, in a case of being viewed in the second direction, an angle formed between a plane direction of the first bottom surface portion and the longitudinal axis direction is 1 degree or less.

5. The ultrasonic endoscope according to claim 2,
wherein the distal end portion includes a balloon groove that is provided between the first region and the second region and that is used for attaching a balloon that covers the ultrasonic transducer.

6. The ultrasonic endoscope according to claim 5,
wherein a groove region of the balloon groove on the other side in the first direction is formed of a pair of groove side surface portions facing each other in the longitudinal axis direction,
a first groove side surface portion that is one of the pair of groove side surface portions is provided on a base end side of the first region in the longitudinal axis direction, and a second groove side surface portion that is the other of the pair of groove side surface portions is provided on a distal end side of the second region in the longitudinal axis direction, and
the first groove side surface portion is formed to extend toward the other side in the first direction with respect to the second groove side surface portion.

7. The ultrasonic endoscope according to claim 1,
wherein the ultrasonic transducer is a convex type in which a plurality of ultrasonic oscillators are arranged in an arc shape along the longitudinal axis direction, and
the elevator is disposed outside an ultrasonic wave scanning range of the ultrasonic transducer.

8. The ultrasonic endoscope according to claim 7,
wherein the ultrasonic wave scanning range of the ultrasonic transducer is 180 degrees or less.
